(19) United States
(12) Patent Application Publication
(10) Pub. No.: US 2026/0125334 A1
(43) Pub. Date: May 7, 2026

(54) TREATMENT PROCESS FOR PFAS-CONTAINING COMPOUNDS

(71) Applicant: Heritage Research Group LLC, Indianapolis, IN (US)

(72) Inventor: Rachel Taylor, Fishers, IN (US)

(73) Assignee: Heritage Research Group, LLC, Indianapolis, IN (US)

(21) Appl. No.: 19/426,452

(22) Filed: Dec. 19, 2025

Related U.S. Application Data
(63) Continuation of application No. PCT/US2024/035609, filed on Jun. 26, 2024.
(60) Provisional application No. 63/510,639, filed on Jun. 28, 2023.

(51) Int. Cl. C07C 17/38; C07C 211/63

(52) U.S. Cl. CPC C07C 17/38; C07C 211/63

(57) ABSTRACT

An improved method for separating PFAS substances from contaminated water and polar solvent systems using alkyl quaternary ammonium compound comprising of coagulation, flocculation, or phase separation thereof is disclosed. The aforementioned compounds may form electrostatic complexes that are insoluble in polar solvents aiding in separation.

### CROSS REFERENCE TO RELATED APPLICATIONS

This application is a continuation of International Application No. PCT/US2024/035609 filed Jun. 26, 2024, which is hereby incorporated herein by reference in its entirety. International Application No. PCT/US2024/035609 claims the benefit of U.S. Provisional Application No. 63/510,639 filed Jun. 28, 2023, which is hereby incorporated herein by reference in its entirety.

### BACKGROUND

Perfluoroalkyl substances and/or polyfluoroalkyl substances (generally referred to as “PFAS”) have been used in consumer products for decades, and because of the strong carbon-fluorine (C—F) bond, these substances do not degrade quickly over time.

Traditionally, flocculants and coagulants have been deployed in efforts to remove PFAS from waste streams, leachates, run-off, and other PFAS-laden liquids, soils, or solids. Some chemical approaches to remove PFAS rely on traditional ferric or aluminum chlorides to remove modest PFAS content while requiring up to twice the standard dosing rate. Furthermore, traditional commercial PFAS flocculants such as polydiallyldimethylammonium chloride (polyDADMAC or polyDDA) and Aluminum chloride are ineffective in solvent systems such as methanol and water.

Therefore, there remains a need for improved treatment processes for removal of PFAS.

### SUMMARY

In various embodiments, methods for removing at least one polyfluoroalkyl or perfluoroalkyl compound from a medium are disclosed. Unless stated differently, such compounds may be referred to as “PFAS” compounds. Generally speaking, the term PFAS shall embody all types of perfluoroalkyl and polyfluoroalkyl species including, but not limited to, perfluoroalkyl sulfonic acids, polyfluoroalkyl sulfonic acids, perfluoroalkanoic acids, and polyfluoroalkanoic acids. The term “perfluoroalkyl” generally refers alkyl substances in which all carbons (except for the last one bound to a functional group) are saturated with fluorine. The term “polyfluoroalkyl” generally refers to alkyl substances that are mostly saturated with fluorines, but also contain carbon-hydrogen bonds. In some embodiments, a quaternary ammonium salt is used. In certain embodiments, a quaternary ammonium salt may comprise the compound of Formula I:

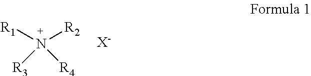

wherein
   R₁ is selected from optionally substituted C₁-C₃₀ alkyl and optionally substituted C₂-C₃₀ alkenyl;
   R₂ is selected from optionally substituted C₁-C₃₀ alkyl and optionally substituted C₂-C₃₀ alkenyl;
   R₃ and R₄ are each independently selected from optionally substituted C₁₀-C₃₀ alkyl and optionally substituted C₁₀-C₃₀ alkenyl; and
   X⁻ is a counter anion.

In some embodiments, the counterion used in Formula I may comprise a halogen. In such embodiments, the halogen may comprise fluoride ion, chloride ion, bromide ion, and/or iodine ion. In other embodiments, a quaternary ammonium salt may comprise the compound of Formula II:

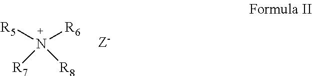

wherein
   R₅ is selected from optionally substituted C₁-C₃₀ alkyl and optionally substituted C₂-C₃₀ alkenyl;
   R₆ is selected from optionally substituted C₁-C₃₀ alkyl and optionally substituted C₂-C₃₀ alkenyl;
   R₇ and R₈ are each independently selected from optionally substituted C₁₀-C₃₀ alkyl and optionally substituted C₁₀-C₃₀ alkenyl; and
      Z⁻ is a perfluoroalkanoate anion, a perfluorosulfonate anion, a polyfluoroalkanoate anion, or a polyfluorosulfonate anion.

In some embodiments, the counterion used in Formula II may comprise a perfluoroalkanoate anion or a perfluorosulfonate anion.

In certain embodiments, the medium comprising at least one PFAS compound for removal comprises sand, soil, and/or water.

In still other embodiments, the compound of Formula I or II may be freely soluble in water, have a solubility of greater than about 1 mg/L of water, or have a solubility of less than 1 mg/L of water. In some embodiments, having a solubility of 1 mg/L or less in water is desirable, as formation of insoluble quaternary perfluoroalkanoate or perfluorosulfonate salts results in the efficient removal of PFAS contaminants.

During processes of removing PFAS from media, an electrostatic complex may be formed. When formed, an electrostatic complex may be insoluble in water. Such electrostatic complex may have a density less than water or have a density greater than that of water.

Additional embodiments of the invention, as well as features and advantages thereof, will be apparent from the descriptions herein.

### BRIEF DESCRIPTION OF THE DRAWINGS

FIG. 1 shows examples of the quaternary ammonium adducts that may form in the presence of anionic functional groups.

FIG. 2 shows the concentration of PFOA and PFOS after treatment with different quaternary amines.

FIG. 3 shows the sum of the concentrations of each PFAS in 10 mL of 1% AFFF solution treated with 100 μL of 5% quat solution.

FIG. 4 shows NMR spectra of AFFF overlayed on AFFF treated with Dimethyldidecylammonium chloride, Dimethyldimyristylammonium Bromide, and Dimethyldioctadecyldecylammonium bromide.

FIG. 5 shows NMR spectra of AFFF solution at different treatment rates of dimethyldimyristylammonium bromide.

FIG. 6 shows NMR total peak area of each solution at different treatment rates of dimethyldimyristylammonium bromide.

FIG. 7 shows a graph plotting the sum of PFAS concentration over time after treatment with dimethyldioctadecylammonium bromide.

FIG. 8 shows a graph of Percent PFAS removal by species.

FIG. 9 shows Bromine concentration in 1000 ppm PFOS and the blank treated with dimethyldioctadecylammonium bromide.

FIG. 10 shows a graph of the PFAS concentration at 0.1% and 0.01% treatment rates in different initial concentrations of PFAS.

### DETAILED DESCRIPTION

For the purposes of promoting an understanding of the principles of the invention, reference will now be made to certain embodiments and specific language will be used to describe the same. It will nevertheless be understood that no limitation of the scope of the invention is thereby intended, such alterations and further modifications, and such further applications of the principles of the invention as described herein being contemplated as would normally occur to one skilled in the art to which the invention relates. Additionally, in the detailed description below, numerous alternatives are given for various features. It will be understood that each such disclosed alternative, or combinations of such alternatives, can be combined with the more generalized features discussed in the Summary above, or set forth in the embodiments described below to provide additional disclosed embodiments herein.

The disclosed invention introduces a novel application of quaternary amines to bind and facilitate removal of PFAS compounds, such as the removal from an aqueous effluent or waste stream. Further, this application affords greater flexibility in the PFAS removal process as it can be deployed independently or as an additive to improve the performance of foam fractionation, phase separation, filtration, precipitation, and other comparable separation techniques

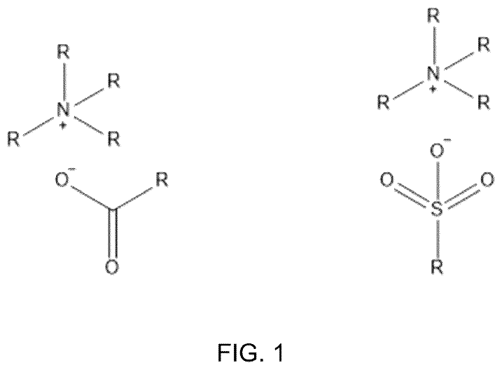

Without being bound by theory, it is believed that the disclosed invention forms a quaternary ammonium adduct, demonstrated in FIG. 1, that aids in separation or coagulation of anionic compounds. Furthermore, the R groups can be tailored for improved function within the desired separation process.

Quaternary ammonium adducts used in embodiments of the present disclosure may comprise quanternary ammonium salts. Radicals on quaternary ammonium adducts may comprise optionally substituted C₁-C₃₀ alkyl and optionally substituted C₂-C₃₀ alkenyl; optionally substituted C₁₀-C₃₀ alkyl; optionally substituted C₁₀-C₃₀ alkenyl; optionally substituted C₁-C₉ alkyl; optionally substituted C₁₂-C₂₀ alkyl; optionally substituted C₂-C₈ alkenyl; and/or optionally substituted C₁₀-C₂₀ alkyl. In some embodiments, the optionally substituted C₁-C₃₀ alkyl radical is methyl, C₁₄ alkyl and/or C₁₈ alkyl. In some embodiments, all of the radicals on quaternary ammonium adducts are unsubstituted.

In some embodiments, the counterion of a quaternary ammonium adduct may comprise a halogen. In such embodiments, the halogen may comprise fluoride ion, chloride ion, bromide ion, and/or iodine ion. In some embodiments, the counterion used may comprise perfluoroalkanoate anion, a perfluorosulfonate anion, a polyfluoroalkanoate anion, or a polyfluorosulfonate anion. In some embodiments, a polyfluoroalkanoate comprises a C₁-C₃₀ alkyl group substituted with at least one fluorine. In some embodiments, a perfluorosulfonate anion comprises a perfluoroalkyl residue, such as, for example, a C₄-C₈ perfluoroalkyl residue.

In certain embodiments, the medium comprising at least one PFAS compound for removal comprises sand, soil, and/or water.

In still other embodiments, the compound of Formula I may be freely soluble in water, have a solubility of greater than about 1 mg/L of water, or have a solubility of less than 1 mg/L of water. In certain embodiments, a medium comprising at least one PFAS compound is contacted with compound of Formula I to remove at least a portion of the at least one PFAS compound by forming a compound of Formula II. In certain embodiments, the compound of Formula II is insoluble or exhibits low solubility in water (e.g., <1 mg/L).

In certain embodiments is described a method of treating a medium comprising a concentration PFAS with an amount of a compound of Formula I. In certain embodiments, the amount of the compound of Formula I is added to reach a target concentration within the medium. In certain embodiments, the compound of Formula I is added to reach a target molar concentration of at least 10 times the molar concentration of PFAS in the medium. In certain embodiments, the compound of Formula I is added to reach a target molar concentration of at least 25 times the molar concentration of PFAS in the medium. In certain embodiments, the compound of Formula I is added to reach a target molar concentration of at least 50 times the molar concentration of PFAS in the medium. In certain embodiments, the compound of Formula I is added to reach a target molar concentration of at least 100 times the molar concentration of PFAS in the medium.

During processes of removing PFAS from media, an electrostatic complex may be formed. When formed, an electrostatic complex may be insoluble in water. Such electrostatic complex may have a density less than water or have a density greater than that of water.

In some embodiments, a PFAS to removed from media may be soluble in water. Without being bound by theory, methods of the present disclosure may result in an ion exchange and the formation of a new salt species.

### EXAMPLES

In order to promote a further understanding of the present invention and its various embodiments, the following specific examples are provided. It will be understood that these examples are illustrative and not limiting of the invention.

### Example 1

Dimethyl quaternary ammonium compounds including dimethyldidecylammonium bromide, dimethyldimyristylammonium bromide, and dimethyloctadidecylammonium bromide, were added to low concentration aqueous perfluorooctanoic acid (PFOA) and perfluorooctane sulfonic acid (PFOS) standard solutions at 0.2% by weight. Precipitate formed in all samples and was removed by centrifugation. The centrate was analyzed by LC-QTOF. Any remaining PFOS and PFOA content in the treated water was identified using LC/QTOF-MS and results are illustrated presented in Table 1. PFAS and PFOA removal improves as chain length of the quaternary ammonium compound increases indicating solubility of the quaternary amine plays an important role in the effectiveness of the flocculant.

TABLE 1
PFOA and PFOS concentration in the presence of dimethyl
quaternary amines with different chain lengths

| | Standard | Dimethyldidecyl- | Dimethyldimyristyl- | Dimethyldioctadecyl- |
|---|---|---|---|---|
| | solution | ammonium | ammonium bromide | ammonium bromide |
| Compound | (ppt) | bromide (ppt) | (ppt) | (ppt) |
| PFOA | 641 | 628 | 149 | 41 |
| PFOS | 818 | 615 | 261 | <5 |

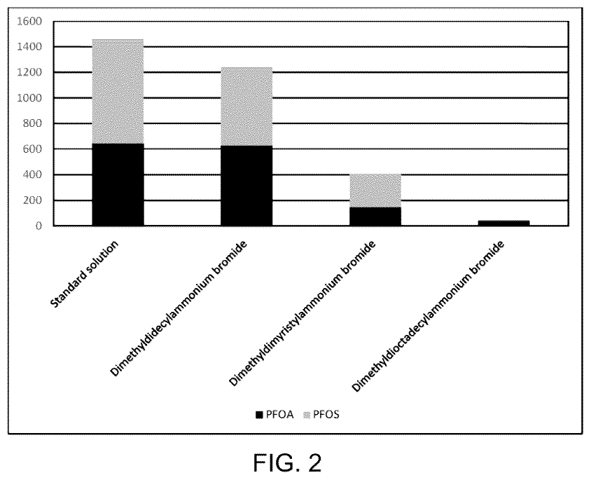

FIG. 2 shows the concentration of PFOA and PFOS after treatment of quaternary amines.

### Example 2

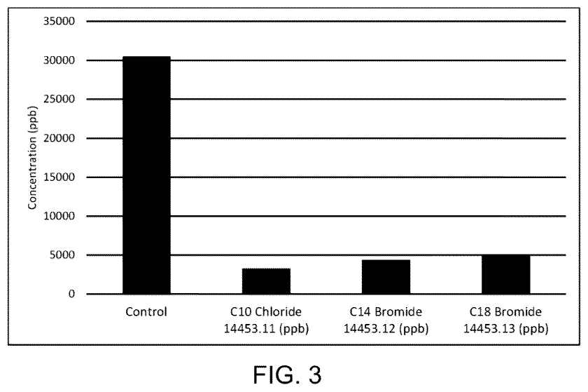

Dimethyldidecylammonium chloride, Dimethyldimyristylammonium Bromide, and Dimethyldioctadecyldecylammonium bromide were each tested in a complex PFAS contaminated sample. The respective quaternary ammonium compound was dissolved/slurried at 5% in equal parts methanol and water by volume. Then, 100 μL of the aforementioned quaternary ammonium compound solution was used to treat 10 mL of synthetic wastewater containing 1% aqueous film forming foam (AFFF). The mixture was homogenized on a vortex mixer for 1 minute. The resulting mixture was centrifuged for 4 minutes at 4000 RPM. The sample separated into a waxy solid precipitate and an aqueous supernatent. The liquid layer from treated and control AFFF samples were analyzed by LC/-QTOF-MS. Results are illustrated in FIG. 3. All three quaternary ammonium compounds tested reduced the PFAS in AFFF contaminated water.

### Example 3

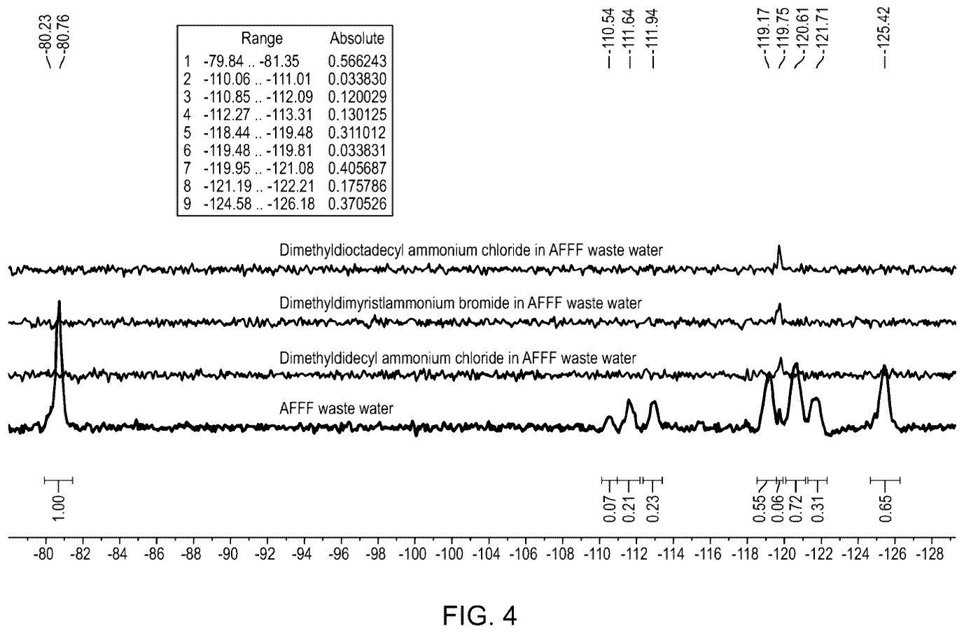

Dimethyldidecylammonium chloride, Dimethyldimyristylammonium Bromide, and Dimethyldioctadecyldecylammonium bromide were each tested in a complex PFAS contaminated sample. The aforementioned quaternary ammonium compound solution was added to a synthetic wastewater produced as a 1.25% AFFF solution in water. The mixture was stirred on a vortex mixer for 1 minute. The resulting mixture was centrifuged for 4 minutes at 4000 RPM. The treated wastewater separated into a waxy solid and an aqueous supernatant. The aqueous layer was analyzed by NMR. FIG. 4 shows the AFFF is not detected in the quaternary ammonium compounds on the NMR spectra. All treated samples produced a peak consistent with the presence of sodium fluoride. The detection limit on the NMR is 100 ppm PFAS.

### Example 4

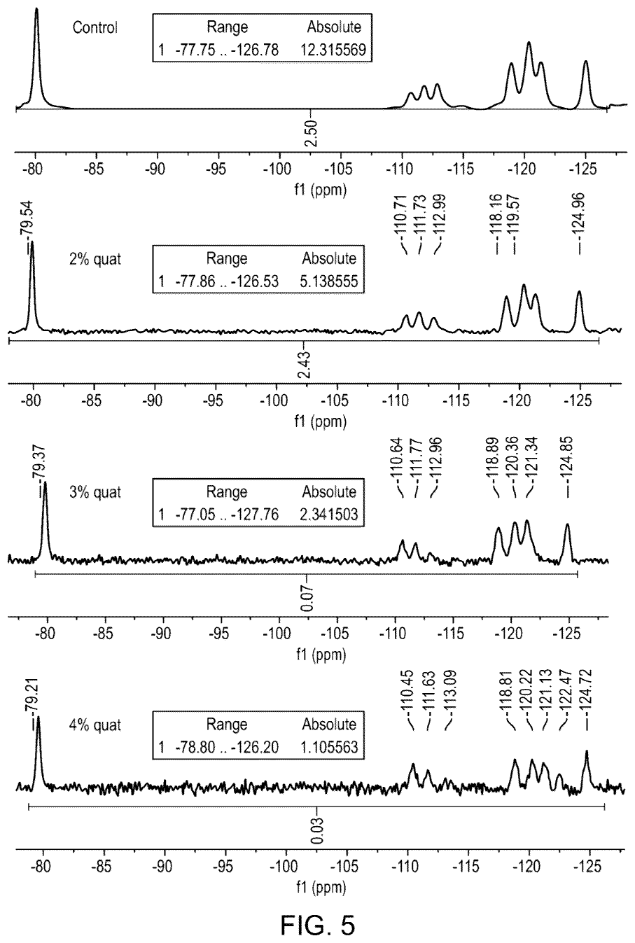

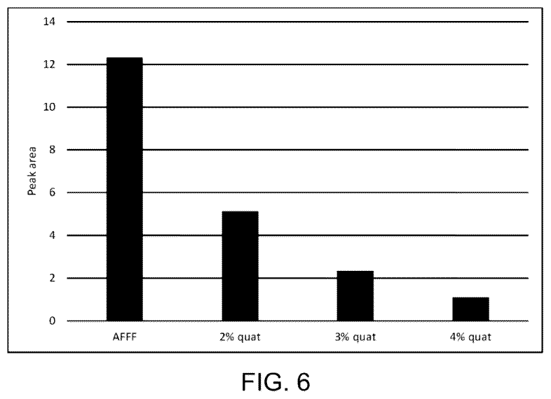

Dimethyldimyristylammonium Bromide was tested in synthetic AFFF wastewater to determine if removal efficiency of the PFAS is affected by treatment rate. The treatment compound was added at 2%, 3%, and 4% w/w loading to AFFF wastewater. Solids were separated out via centrifugation and the solution was analyzed by NMR. As the concentration of the quaternary ammonium compound increased, the concentration of remaining PFAS decreased in the AFFF wastewater. FIG. 5 is the NMR spectra of each solution after treatment. FIG. 6 shows the sum of the peak areas at different concentrations of quat loading. As quat loading increased, the total area under the peaks was reduced. In the sample with 4% dimethyldioctadecylammonium bromide loading, total peak area was reduced by 90%.

### Example 5

A standard with approximately 1000 ppt each of PFOA, perfluoro-n-pentanoic acid (PFPeA), and perfluorobutane sulfonic acid (PFBS) was prepared via serial dilution and treated with 2% dimethyldioctadecylammonium bromide in technical triplicate. Samples were centrifuged and the liquid was filtered into a new centrifuge tube using a 0.45 micron syringe filter.

Table 2 indicates the concentration of each PFAS after treatment. There was a significant reduction in all PFAS concentrations by >99.87%.

TABLE 2
Concentration of short chain PFAS after treatment
with 2% dimethyldioctadecylammonium bromide.

| | | Average PFAS after |
|---|---|---|
| Compound | Control (ppt) | treatment (ppt) |
| PFPeA | 1419 | <5 |
| PFBS | 1917 | ND |
| PFOA | 563 | ND |

ND: Non-detect.

### Example 6

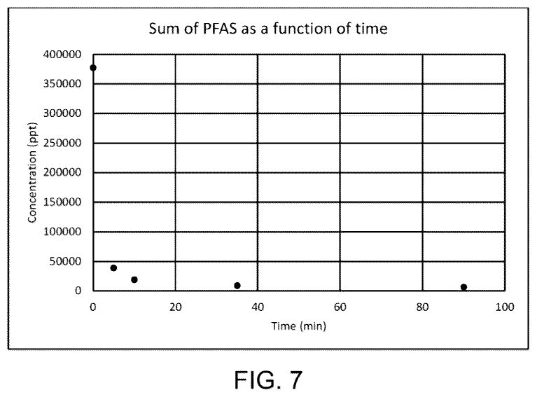

To determine PFAS removal kinetics, a PFAS-contaminated field sample was treated with dimethyldioctadecylammonium bromide. Aliquots were pulled at timed intervals and tested for remaining PFAS content. To a polypropylene bottle, 1000 mL of approximately 4 ppm PFAS-contaminated water was added to a 1 L HDPE bottle with a Teflon stir bar. The sample was stirred at 500 RPM for 10 minutes prior to the start of the experiment. To a 50 mL centrifuge tube, 20 mL aliquots were collected at 0, 5, 10, 30, and 90 minutes following treatment with 0.01%. Each sample was centrifuged to remove any remaining solids and passed through 0.45 micron filter prior to analysis by LC-QTOF. FIG. 7 shows the data collected from the kinetics trial. The data indicates a rapid decrease in remaining PFAS content between the T=0 and T=5 sample. Subsequent PFAS removal was noted but minimal.

### Example 7

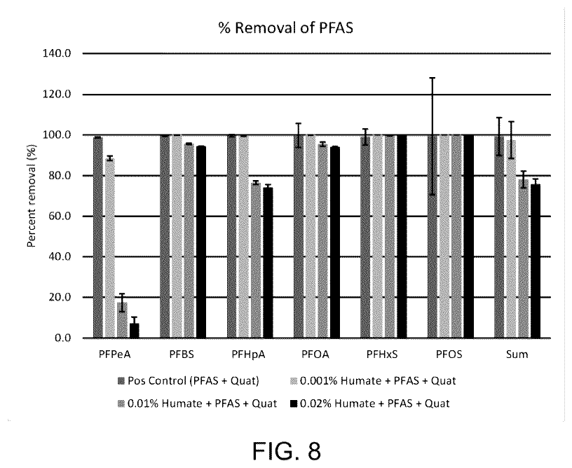

To determine how PFAS removal is affected by competing contaminants, sodium humate was added to a solution with 10 ppb, each, of PFOA, PFOS, perfluoroheptanoic acid (PFHpA), perfluorohexane sulfonic acid (PFHxS), PFPeA, and perfluorobutanoic sulfonate (PFBS). Sodium humate was added to yield, four tubes each, with contaminate concentrations of 0%, 0.001%, 0.01%, and 0.02%. 100 mg (0.2%) of dimethyldioctadecylammonium bromide was added to three of each set of samples. Samples were mixed for 30 seconds and centrifuged for 30 minutes. Samples were filtered through a 0.45 micron PES filter and LC was run. FIG. 8 shows a graph of percent PFAS removal by species. As sodium humate concentration increased, the PFAS removal decreased in PFPeA and PFHpA. PFPeA was more influenced by the sodium humate than the other PFAS compounds.

### Example 8

In effort to understand the PFAS removal mechanism, an experiment was developed to track bromide in the solution before and after treatment. If there is an ion exchange interaction, it would be expected that the bromine concentration in the treated sample would be elevated compared to the control. To a polypropylene container, 100 mL of 1000 ppm PFOS and 100 mL of MilliQ water (blank) was treated with 2 g dimethyldioctadecylammonium bromide. Additionally, 100 mL of 1000 ppm PFOS was left as a control. Samples were repeated in triplicate. All samples were filtered through a 0.45 micron cellulose filter via gravity filtration. Liquid samples were analyzed for bromine by XRF.

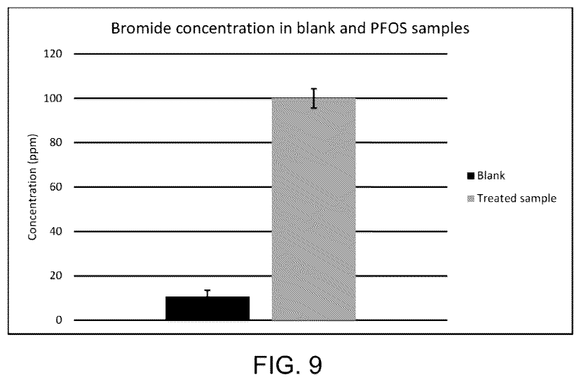

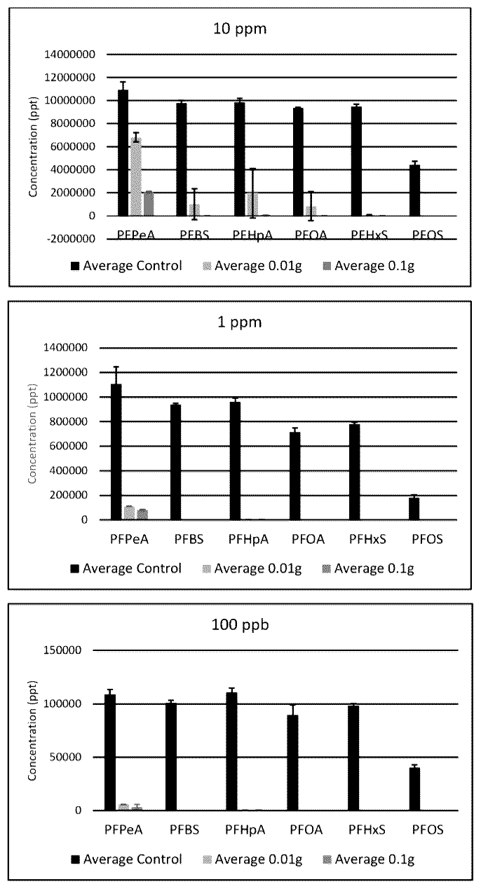

FIG. 9 shows the difference in bromine concentration in a water solution after treating a blank and a 1000 ppm PFOS sample with dimethyldioctadecylammonium bromide. The increase in bromine in the PFOS sample indicates there is an ion exchange interaction with the quaternary ammonium compound and the PFAS species. No significant increase in bromine was seen in the blank treated only with dimethyldioctadecylammonium bromide, which is nearly insoluble in water.

### Example 9

To understand the optimal treatment rates for different initial PFAS concentration, a concentration study was conducted. Standards containing 10 ppm, 1 ppm, 100 ppb, 10 ppb, and 1 ppb each of six PFAS species was treated with 0.1% and 0.01% of dimethyldioctadecylammonium bromide. Samples were agitated on a vortex mixer for 30 seconds and mixed on an orbishaker for 1 hour. Approximately 20 mL of each sample was filtered into a centrifuge tube using a 0.45 micron PES filter. Each Sample was analyzed by LC-MS to quantify the PFAS present.

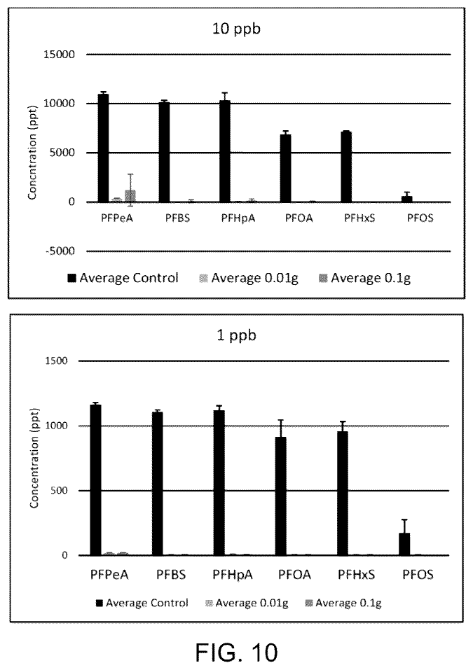

FIG. 10 shows the PFAS concentration in different initial concentrations of PFAS. As the concetratoin of quat increased in relation to the PFAS, removal efficiency increased. The optimal treatment rate was determined to be a 100x molar excess of dimethyldioctadecylammonium bromide.

The uses of the terms “a” and “an” and “the” and similar references in the context of describing the invention (especially in the context of the following claims) are to be construed to cover both the singular and the plural unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

While the invention has been illustrated and described in detail in the drawings and the foregoing description, the same is to be considered as illustrative and not restrictive in character, it being understood that only the preferred embodiment has been shown and described and that all changes and modifications that come within the spirit of the invention are desired to be protected. In addition, all references cited herein are indicative of the level of skill in the art and are hereby incorporated by reference in their entirety.

### EMBODIMENTS

The following provides an enumerated listing of some of the embodiments disclosed herein. It will be understood that this listing is non-limiting, and that individual features or combinations of features (e.g. 2, 3 or 4 features) as described in the Detailed Description above can be incorporated with the below-listed Embodiments to provide additional disclosed embodiments herein.

1. A method of removing at least one perfluoroalkyl or polyfluoroalkyl compound from a medium, comprising contacting said medium with a compound of Formula I:

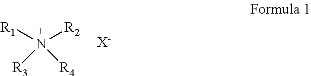

wherein
   R₁ is selected from optionally substituted C₁-C₃₀ alkyl and optionally substituted C₂-C₃₀ alkenyl;
   R₂ is selected from optionally substituted C₁-C₃₀ alkyl and optionally substituted C₂-C₃₀ alkenyl;
   R₃ and R₄ are each independently selected from optionally substituted C₁₀-C₃₀ alkyl and optionally substituted C₁₀-C₃₀ alkenyl; and
   X⁻ is a counter anion.

2. The method of embodiment 1, wherein R₁ is selected from optionally substituted C₁-C₉ alkyl and optionally substituted C₂-C₈ alkenyl.

3. The method of embodiment 2, wherein R₁ is methyl.

4. The method of any of the preceding embodiments, wherein R₂ is selected from optionally substituted C₁-C₉ alkyl and optionally substituted C₂-C₈ alkenyl.

5. The method of claim 4, wherein R₂ is methyl.

6. The method of any of the preceding embodiments, wherein R₃ and R₄ are independently selected from optionally substituted C₁₀-C₂₀ alkyl.

7. The method of any of the preceding embodiments, wherein R₁, R₂, R₃ and R₄ are all unsubstituted.

8. The method of any of the preceding embodiments, wherein the counter anion is a halogen anion.

9. The method of embodiment 8, wherein the counter anion is a chloride or bromide anion.

10. The method of any of the preceding embodiments, wherein the medium comprises sand or soil.

11. The method of any of the preceding embodiments, wherein the medium comprises water.

12. The method of any of the preceding embodiments, wherein contacting the polyfluoroalkyl compound with the compound of Formula I results in the formation of an electrostatic complex.

13. The method of embodiment 12, wherein the electrostatic complex is insoluble in water.

14. The method of embodiment 13, wherein the electrostatic complex is less dense than water.

15. The method of embodiment 13, wherein the electrostatic complex is more dense than water.

16. The method of any of the preceding embodiments, wherein the polyfluoroalkyl compound is soluble in water.

17. The method of any of the preceding embodiments, wherein the compound of Formula I is soluble in water.

18. The method of any of any of embodiments 1-16, wherein the compound of Formula I exhibits a solubility of greater than about 1 mg/L of water.

19. The method of any one of embodiments 1-16, wherein the compound of Formula I exhibits a solubility of less than 1 mg/L of water.

20. The method of any one of the preceding embodiments, wherein contacting the perfluoroalkyl compound or polyfluoroalkyl compound with the compound of Formula I results in ion exchange and the formation of a new salt species.

21. The method of any one of the preceding embodiments, wherein X- is a halogen anion.

22. The method of embodiment 21, wherein X- is a bromine anion.

23. The method of any of embodiments 20-22, wherein the new salt species is selected from Formula II:

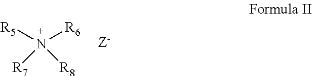

wherein
   R₅ is selected from optionally substituted C₁-C₃₀ alkyl and optionally substituted C₂-C₃₀ alkenyl;
   R₆ is selected from optionally substituted C₁-C₃₀ alkyl and optionally substituted C₂-C₃₀ alkenyl;
   R₇ and R₈ are each independently selected from optionally substituted C₁₀-C₃₀ alkyl and optionally substituted C₁₀-C₃₀ alkenyl; and
      Z⁻ is a perfluoroalkanoate anion, a perfluorosulfonate anion, a polyfluoroalkanoate anion, or a polyfluorosulfonate anion.

24. A compound of Formula II:

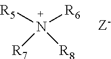

wherein
   R₅ is selected from optionally substituted C₁-C₃₀ alkyl and optionally substituted C₂-C₃₀ alkenyl;
   R₆ is selected from optionally substituted C₁-C₃₀ alkyl and optionally substituted C₂-C₃₀ alkenyl;
   R₇ and R₈ are each independently selected from optionally substituted C₁₀-C₃₀ alkyl and optionally substituted C₁₀-C₃₀ alkenyl; and
      Z⁻ is a perfluoroalkanoate anion, a perfluorosulfonate anion, a polyfluoroalkanoate anion, or a polyfluorosulfonate anion.

25. The compound of embodiment 24, wherein R₅ is selected from optionally substituted C₁-C₉ alkyl and optionally substituted C₂-C₈ alkenyl.

26. The compound of embodiment 25, wherein R₅ is methyl.

27. The compound of any one of embodiments 24-26, wherein R₆ is selected from optionally substituted C₁-C₉ alkyl and optionally substituted C₂-C₈ alkenyl.

28. The compound of claim 27, wherein R₆ is methyl.

29. The compound of any one of embodiments 24-28, wherein R₇ and R₈ are independently selected from optionally substituted C₁₀-C₂₀ alkyl.

30. The compound of any one of embodiments 24-29, wherein R₅, R₆, R₇ and R₈ are all unsubstituted.

31. The compound of any one of embodiments 24-30, wherein Z is selected from Formula IIIa and IIIb:

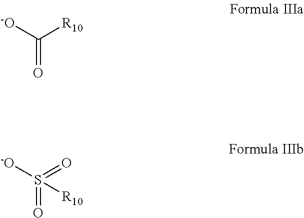

wherein R₁₀ is a C₁-C₃₀ alkyl group substituted with at least one fluorine.

32. The compound of embodiment 31, wherein R₁₀ is a perfluoroalkyl residue.

33. The compound of embodiment 32, wherein R₁₀ is a C₄-C₈ perfluoroalkyl residue.

34. The method according to any one of embodiments 1-22, wherein R₃ is selected from optionally substituted C₁₂-C₂₀ alkyl.

35. The method of embodiment 34, wherein R₄ is selected from C₁₂-C₂₀ alkyl.

36. The method of any one of embodiments 34-35, wherein at least one of R₃ and R₄ is selected from C₁₄ alkyl and C₁₈ alkyl.

37. The compound according to any one of embodiments 24-33, wherein R₇ is selected from optionally substituted C₁₂-C₂₀ alkyl.

38. The method of embodiment 37, wherein R₈ is selected from C₁₂-C₂₀ alkyl.

39. The method of any one of embodiments 37-38, wherein at least one of R₇ and R₈ is selected from C₁₄ alkyl and C₁₈ alkyl.

40. A method of treating a medium containing an initial molar concentration of a perfluoroalkyl compound or a polyfluoroalkyl compound, comprising contacting said medium a compound of Formula I:

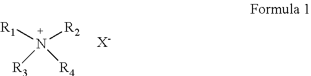

wherein
   R₁ is selected from optionally substituted C₁-C₃₀ alkyl and optionally substituted C₂-C₃₀ alkenyl;
   R₂ is selected from optionally substituted C₁-C₃₀ alkyl and optionally substituted C₂-C₃₀ alkenyl;
   R₃ and R₄ are each independently selected from optionally substituted C₁₀-C₃₀ alkyl and optionally substituted C₁₀-C₃₀ alkenyl; and
   X⁻ is a counter anion.

41. The method of embodiment 40, wherein the medium is contacted with an amount of the compound of Formula I sufficient to create a molar concentration in the medium of said compound of Formula I that is at least 10 times greater than the initial molar concentration of the perfluoroalkyl compound or the polyfluoroalkyl compound in the medium.

42. The method of embodiment 41, wherein the medium is contacted with an amount of the compound of Formula I sufficient to create a molar concentration in the medium of said compound of Formula I that is at least 50 times greater than the initial molar concentration of the perfluoroalkyl compound or the polyfluoroalkyl compound in the medium.

43. The method of embodiment 41, wherein the medium is contacted with an amount of the compound of Formula I sufficient to create a molar concentration in the medium of said compound of Formula I that is at least 100 times greater than the initial molar concentration of the perfluoroalkyl compound or the polyfluoroalkyl compound in the medium.

44. The method of any one of embodiments 40-43, wherein contacting the medium with the compound of Formula I yields a final molar concentration of the perfluoroalkyl compound or the polyfluoroalkyl compound that is at least 90% lower than the initial molar concentration.

45. The method of any one of embodiments 40-43, wherein contacting the medium with the compound of Formula I yields a final concentration of the perfluoroalkyl compound or the polyfluoroalkyl compound that is at least 95% lower than the initial concentration.

46. The method of any one of embodiments 40-43, wherein contacting the medium with the compound of Formula I yields a final concentration of the perfluoroalkyl compound or the polyfluoroalkyl compound that is at least 98% lower than the initial concentration.

1. A method of removing at least one polyfluoroalkyl compound from a medium, comprising contacting said medium with a compound of Formula I: 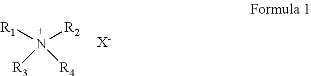
wherein
R₁ is selected from optionally substituted C₁-C₃₀ alkyl and optionally substituted C₂-C₃₀ alkenyl;
R₂ is selected from optionally substituted C₁-C₃₀ alkyl and optionally substituted C₂-C₃₀ alkenyl;
R₃ and R₄ are each independently selected from optionally substituted C₁₀-C₃₀ alkyl and optionally substituted C₁₀-C₃₀ alkenyl; and
X⁻ is a counter anion.

2. The method of claim 1, wherein R₁ is selected from optionally substituted C₁-C₉ alkyl and optionally substituted C₂-C₈ alkenyl.

3. The method of claim 2, wherein R₁ is methyl.

4. The method of claim 2, wherein R₂ is selected from optionally substituted C₁-C₉ alkyl and optionally substituted C₂-C₈ alkenyl.

5. The method of claim 4, wherein R₂ is methyl.

6. The method of claim 1, wherein R₃ and R₄ are independently selected from optionally substituted C₁₀-C₂₀ alkyl.

7. The method of claim 1, wherein R₁, R₂, R₃ and R₄ are all unsubstituted.

8. The method of claim 1, wherein the counter anion is a halogen anion.

9. The method of claim 8, wherein the counter anion is a chloride or bromide anion.

10. The method of claim 1, wherein the medium comprises sand or soil.

11. The method of claim 1, wherein the medium comprises water.

12. The method of claim 1, wherein contacting the polyfluoroalkyl compound with the compound of Formula I results in the formation of an electrostatic complex.

13. The method of claim 12, wherein the electrostatic complex is insoluble in water.

14. The method of claim 13, wherein the electrostatic complex is less dense than water.

15. The method of claim 13, wherein the electrostatic complex is more dense than water.

16. The method of claim 1, wherein the polyfluoroalkyl compound is soluble in water.

17. The method of claim 1, wherein the compound of Formula I is soluble in water.

18. The method of claim 1, wherein the compound of Formula I exhibits a solubility of greater than about 1 mg/L of water.

19. The method of claim 1, wherein the compound of Formula I exhibits a solubility of less than 1 mg/L of water.

20. The method of claim 1, wherein contacting the polyfluoroalkyl compound with the compound of Formula I results in ion exchange and the formation of a new salt species.

21. The method of claim 1, wherein X- is a halogen anion.

22. The method of claim 21, wherein X- is a bromine anion.

23. The method of claim 20, wherein the new salt species is selected from Formula II: 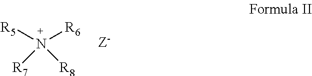
wherein
R₅ is selected from optionally substituted C₁-C₃₀ alkyl and optionally substituted C₂-C₃₀ alkenyl;
R₆ is selected from optionally substituted C₁-C₃₀ alkyl and optionally substituted C₂-C₃₀ alkenyl;
R₇ and R₈ are each independently selected from optionally substituted C₁₀-C₃₀ alkyl and optionally substituted C₁₀-C₃₀ alkenyl; and
Z⁻ is a perfluoroalkanoate anion, a perfluorosulfonate anion, a polyfluoroalkanoate anion,
or a polyfluorosulfonate anion.

24. A compound of Formula II: 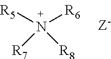
wherein
R₅ is selected from optionally substituted C₁-C₃₀ alkyl and optionally substituted C₂-C₃₀ alkenyl;
R₆ is selected from optionally substituted C₁-C₃₀ alkyl and optionally substituted C₂-C₃₀ alkenyl;
R₇ and R₈ are each independently selected from optionally substituted C₁₀-C₃₀ alkyl and optionally substituted C₁₀-C₃₀ alkenyl; and
Z⁻ is a perfluoroalkanoate anion, a perfluorosulfonate anion, a polyfluoroalkanoate anion, or a polyfluorosulfonate anion.

25. The compound of claim 24, wherein R₅ is selected from optionally substituted C₁-C₉ alkyl and optionally substituted C₂-C₈ alkenyl.

26. The compound of claim 25, wherein R₅ is methyl.

27. The compound of claim 24, wherein R₆ is selected from optionally substituted C₁-C₉ alkyl and optionally substituted C₂-C₈ alkenyl.

28. The compound of claim 27, wherein R₆ is methyl.

29. The compound of claim 24, wherein Ry and R₈ are independently selected from optionally substituted C₁₀-C₂₀ alkyl.

30. The compound of claim 24, wherein R₅, R₆, R₇ and R₈ are all unsubstituted.

31. The compound of claim 24, wherein Z⁻ is selected from Formula Illa and IIIb: 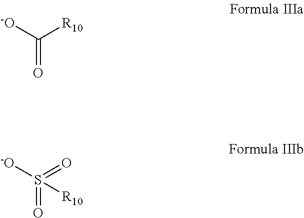
wherein R₁₀ is a C₁-C₃₀ alkyl group substituted with at least one fluorine.

32. The compound of claim 31, wherein R₁₀ is a perfluoroalkyl residue.

33. The compound of claim 32, wherein R₁₀ is a C₄-C₈ perfluoroalkyl residue.

34. The method according to claim 1, wherein R₃ is selected from optionally substituted C₁₂-C₂₀ alkyl.

35. The method of claim 34, wherein R₄ is selected from C₁₂-C₂₀ alkyl.

36. The method of claim 34, wherein at least one of R₃ and R₄ is selected from C₁₄ alkyl and C₁₈ alkyl.

37. The compound according to claim 24, wherein R₇ is selected from optionally substituted C₁₂-C₂₀ alkyl.

38. The method of claim 37, wherein R₈ is selected from C₁₂-C₂₀ alkyl.

39. The method of claim 37, wherein at least one of R₇ and R₈ is selected from C₁₄ alkyl and C₁₈ alkyl.

40. A method of treating a medium containing an initial molar concentration of a perfluoroalkyl compound or a polyfluoroalkyl compound, comprising contacting said medium a compound of Formula I: 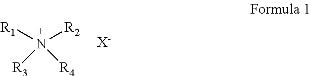
wherein
R₁ is selected from optionally substituted C₁-C₃₀ alkyl and optionally substituted C₂-C₃₀ alkenyl;
R₂ is selected from optionally substituted C₁-C₃₀ alkyl and optionally substituted C₂-C₃₀ alkenyl;
R₃ and R₄ are each independently selected from optionally substituted C₁₀-C₃₀ alkyl and optionally substituted C₁₀-C₃₀ alkenyl; and
X⁻ is a counter anion.

41. The method of claim 40, wherein the medium is contacted with an amount of the compound of Formula I sufficient to create a molar concentration in the medium of said compound of Formula I that is at least 10 times greater than the initial molar concentration of the perfluoroalkyl compound or the polyfluoroalkyl compound in the medium.

42. The method of claim 41, wherein the medium is contacted with an amount of the compound of Formula I sufficient to create a molar concentration in the medium of said compound of Formula I that is at least 50 times greater than the initial molar concentration of the perfluoroalkyl compound or the polyfluoroalkyl compound in the medium.

43. The method of claim 41, wherein the medium is contacted with an amount of the compound of Formula I sufficient to create a molar concentration in the medium of said compound of Formula I that is at least 100 times greater than the initial molar concentration of the perfluoroalkyl compound or the polyfluoroalkyl compound in the medium.

44. The method of claim 40, wherein contacting the medium with the compound of Formula I yields a final molar concentration of the perfluoroalkyl compound or the polyfluoroalkyl compound that is at least 90% lower than the initial molar concentration.

45. The method of claim 40, wherein contacting the medium with the compound of Formula I yields a final concentration of the perfluoroalkyl compound or the polyfluoroalkyl compound that is at least 95% lower than the initial concentration.

46. The method of claim 40, wherein contacting the medium with the compound of Formula I yields a final concentration of the perfluoroalkyl compound or the polyfluoroalkyl compound that is at least 98% lower than the initial concentration.
